(19) United States
(12) Patent Application Publication
(10) Pub. No.: US 2024/0091209 A1
(43) Pub. Date: Mar. 21, 2024

(54) COMPOSITIONS AND METHODS FOR TREATMENT OF HIDRADENITIS SUPPURATIVA

(71) Applicant: SUZHOU KINTOR PHARMACEUTICALS, INC., Suzhou (CN)

(72) Inventors: Ofer A. GOREN, Irvine, CA (US); John MCCOY, Irvine, CA (US)

(73) Assignee: SUZHOU KINTOR PHARMACEUTICALS, INC., Suzhou (CN)

(21) Appl. No.: 18/264,143

(22) PCT Filed: Jan. 28, 2022
(86) PCT No.: PCT/CN2022/074460

§ 371 Date: Aug. 3, 2023

Related U.S. Application Data
(60) Provisional application No. 63/145,731, filed on Feb. 4, 2021.

(51) Int. Cl. A61K 31/4439; A61K 31/155; A61K 31/566; A61P 17/00

(52) U.S. Cl. CPC A61K 31/4439; A61K 31/155; A61K 31/566; A61P 17/00

(57) ABSTRACT

Compositions, methods and uses are disclosed herein for treating male and female hidradenitis suppurativa with anti-androgen medications. Clinical examples of treatment of hidradenitis suppurativa with proxalutamide are provided. The methods, uses and composition described can be applied as treatment to mitigate an outbreak of hidradenitis suppurativa or as a prophylactic to prevent the occurrence of hidradenitis suppurativa.

This application claims the priority of U.S. Patent Application No. 63/145,731, filed on Feb. 4, 2020 and titled with “Compositions and Methods for Treatment of Hidradenitis Suppurativa” and the disclosures of which are hereby incorporated by reference.

### FIELD

The present invention relates to compositions and methods for the treatment of hidradenitis suppurativa with anti-androgen medications.

### BACKGROUND

Hidradenitis suppurativa (HS) also known as acne inversa is a chronic skin disease. It causes painful nodules, abscesses, and boil-like lumps, which manifests under apocrine gland-rich skin. It often affects areas of the body where the skin rubs together, such as armpits and the groin. Nodules can become ruptured causing abscesses that drain fluid and pus. As the abscesses heal, they can cause scarring.

Medical practitioners have noted antidotally that HS appears after puberty and flares during the pre-menstrual cycle in females. Additionally, it has been noted that the condition improves with pregnancy and after menopause. These changes in HS cycles indicate that hormones play a role in the pathogenesis of HS. Although this connection to hormones has been inferred, clinical evidence supporting this hypothesis are lacking. The only randomized clinical trial published to date was conducted in 1986 by Mortimer, et al. (See, Mortimer, Dawber, Gales, & Moore. Br J Dermatol. 115(3): 263-268 (1986)).

Mortimer et al. reported anti-androgen treatment of 24 female patients with HS. Subjects were treated with ethinyloestradiol (50 μg) and cyproterone acetate (50 mg) compared to ethinyloestradiol (50 μg) and norgestrel (500 μg) (Eugynon 50). They observed positive benefits in both groups but reported no statistically significant advantage to the cyproterone acetate (an antiandrogen). Although they concluded that antiandrogen therapy appeared to be beneficial for HS, definitive clinical proof that antiandrogen therapy is efficacious for HS is still lacking.

Many antiandrogen drugs are only indicated for men and carry warnings prohibiting use in women. Proxalutamide (GT0918) is a novel second generation androgen receptor antagonist that has several distinct advantages as a potential therapy for HS. It is highly effective antagonist of AR as well as exhibiting pharmacological effects of inducing the down-regulation of AR expression. This second mechanism is not present in similar drug of this class, for example, bicalutamide or enzalutamide. Because of the dual mechanism of action, it is expected to be a more effective and less toxic second-generation anti-androgen drug therapy. Clinical evidence has demonstrated that proxalutamide lowers AR expression and activity. Proxalutamide has been show safe in both men and women in Phase II studies.

No approved medical treatments for HS currently exist. As such, a novel clinically validated treatment would be a welcome addition.

### SUMMARY

Compositions, methods and uses are disclosed herein for treating male and female hidradenitis suppurativa with anti-androgen medications. Clinical examples enabling the compositions, methods and uses are provided. The methods, uses and composition described can be applied as treatment to mitigate an outbreak of hidradenitis suppurativa or as a prophylactic to prevent the occurrence of hidradenitis suppurativa.

Certain embodiments presented within the application relate to a method for treating or preventing hidradenitis suppurativa in a subject in need thereof, wherein the method comprises administering a therapeutically effective amount of an anti-androgen composition to the subject, wherein the composition comprises proxalutamide.

In some embodiments, the method comprises administering a therapeutically effective amount of an anti-androgen composition comprising proxalutamide to a subject, wherein the composition further comprises other anti-androgens selected from the group consisting of androgen receptor antagonists, androgen synthesis inhibitors, sex hormone-binding globulin (SHBG) stimulators, antigonadotropins, mineralocorticoids, glucocorticoids, insulin sensitizing medications, and vaccines or immunogens against androstenedione.

In other embodiments, the method comprises administering a therapeutically effective amount of an anti-androgen composition comprising proxalutamide to a subject, wherein the composition further comprises glucocorticoids and mineralocorticoids such as anticorticotropin.

In some embodiments, the method comprises administering a therapeutically effective amount of an anti-androgen composition comprising proxalutamide to a subject, wherein the composition further comprises insulin sensitizing medication such as metformin.

In other embodiments, the method comprises administering a therapeutically effective amount of an anti-androgen composition comprising proxalutamide to a subject, wherein the composition further comprises vaccines or immunogens against androstenedione such as ovandrotone albumin and androstenedione albumin.

In some embodiments, the method comprises administering a therapeutically effective amount of an anti-androgen composition comprising proxalutamide to a subject, wherein the composition further comprises other anti-androgens in amounts ranging from 0.1% to 99.9% of the total weight of the composition.

In other embodiments, the method comprises administering a therapeutically effective amount of an anti-androgen composition comprising proxalutamide to a subject, wherein proxalutamide is present in an amount ranging from 0.1% to 50% of the total weight of the composition.

In some embodiments, the method comprises administering a therapeutically effective amount of an anti-androgen composition consisting essentially of proxalutamide to a subject.

In other embodiments, the method comprises administering a therapeutically effective amount of an anti-androgen composition comprising proxalutamide to a subject, wherein the method further comprises determining if the subject is sensitive to anti-androgen treatment by examining an occurrence of genetic variations in the subject's androgen receptor gene or in the subject's androgen response elements of other genes under regulatory control of the androgen receptor.

In some embodiments, the method comprises administering a therapeutically effective amount of an anti-androgen composition comprising proxalutamide to a subject, wherein the method further comprises determining if the subject is sensitive to anti-androgen treatment by examining an occurrence of genetic variations in the subject's androgen receptor gene or in the subject's androgen response elements of other genes under regulatory control of the androgen receptor, wherein the genetic variations occur in the number CAG repeats in the first exon of the androgen receptor gene, the number GGN repeats in the first exon of the androgen receptor gene or in the promoter region of the androgen receptor gene.

In other embodiments, the method comprises administering a therapeutically effective amount of an anti-androgen composition comprising proxalutamide to a subject, wherein the method further comprises determining if the subject is sensitive to anti-androgen treatment by examining an occurrence of genetic variations in the subject's androgen receptor gene or in the subject's androgen response elements of other genes under regulatory control of the androgen receptor, wherein the genetic variation occurs in the subject's androgen response elements for TMPRSS2, furin and ACE2.

In some embodiments, the method comprises administering a therapeutically effective amount of an anti-androgen composition comprising proxalutamide to a subject, wherein the method further comprises determining if the subject is sensitive to anti-androgen treatment by examining an occurrence of genetic variations in the subject's androgen receptor gene or in the subject's androgen response elements of other genes under regulatory control of the androgen receptor, wherein the occurrence of genetic variations are in the androgen receptor gene or promoter region of the androgen receptor gene, and wherein the genetic variations are rs137852591, rs104894742, rs1057518177, rs1057521121, rs1057521122, rs1057523747, rs1064793480, rs1064793645, rs1064794065, rs1064794069, rs1064795250, rs1085307685, rs1085307962, rs12014709, rs1204038, rs1337080, rs137852562, rs137852563, rs137852564, rs137852565, rs137852566, rs137852567, rs137852568, rs137852569, rs137852570, rs137852571, rs137852572, rs137852573, rs137852574, rs137852575, rs137852576, rs137852577, rs137852578, rs137852579, rs137852580, rs137852581, rs137852582, rs137852583, rs137852584, rs137852585, rs137852586, rs137852587, rs137852588, rs137852589, rs137852590, rs137852592, rs137852593, rs137852594, rs137852595, rs137852596, rs137852597, rs137852598, rs137852599, rs137852600, rs137852601, rs1800053, rs201934623, rs2361634, rs5031002, rs5918757, rs6152, rs6624304, rs750324117, rs754201976, rs755226547, rs759327087, rs864622007, rs869320731, rs869320732, rs878853033, rs886039558, rs886041050, rs886041128, rs886041129, rs886041130, rs886041131, rs886041132, rs886041133, rs886041352, rs9332969, rs9332971 or any combination thereof.

In other embodiments, the method comprises administering a therapeutically effective amount of an anti-androgen composition comprising proxalutamide to a subject, wherein the amount of proxalutamide in the composition ranges from 50-400 mgs.

In some embodiments, the method comprises administering a therapeutically effective amount of an anti-androgen composition comprising proxalutamide to a subject, wherein the composition is administered topically as a gel, foam or lotion into a HS lesion on the subject.

In other embodiments, the method comprises administering a therapeutically effective amount of an anti-androgen composition comprising proxalutamide to a subject, wherein the composition is administered orally to the subject.

In some embodiments, the method comprises administering a therapeutically effective amount of an anti-androgen composition comprising proxalutamide to a subject, wherein the composition is administered in an amount ranging from 0.5 mg to 800 mg to the subject.

Certain embodiments presented within the application relate to an anti-androgen composition for treating or preventing HS in a subject in need thereof, wherein the composition comprises a therapeutically effective amount proxalutamide.

In some embodiments, the anti-androgen composition comprises a therapeutically effective amount proxalutamide with an anti-inflammatory agent and/or an anti-bacterial agent.

In other embodiments, the anti-androgen composition comprises a therapeutically effective amount proxalutamide with other anti-androgens selected from the group consisting of androgen receptor antagonists, androgen synthesis inhibitors, sex hormone-binding globulin (SHBG) stimulators, antigonadotropins, mineralocorticoids, glucocorticoids, insulin sensitizing medications, and vaccines or immunogens against androstenedione.

In some embodiments, the anti-androgen composition comprises a therapeutically effective amount proxalutamide with aspartame.

In other embodiments, the anti-androgen composition comprises a therapeutically effective amount proxalutamide with carriers or delivery vehicles selected from capsules liposomes, non-ionic liposomes, niosomes, novasome I, erythromycin-Zn complex, microspheres, nanoparticles, solid lipid nanoparticles and nanoemulsions.

In some embodiments, the anti-androgen composition comprises a therapeutically effective amount proxalutamide with agents that promote the production of estrogen, wherein the agents are selected from estrogen, estradiol, conjugated estrogens, medroxyprogesterone acetate, Provera and medroxyprogesterone.

Corresponding to all of the above-mentioned aspects of the method, the present disclosure also provides use of an anti-androgen composition in the manufacture of a medicament for treating or preventing hidradenitis suppurativa in a subject in need thereof, wherein the composition comprises proxalutamide.

Certain embodiments presented within the application relate to a kit for treating or preventing HS in a subject in need thereof, wherein the kit comprises a DNA collection device and a DNA diagnostic assay.

In some embodiments, the kit comprises a DNA collection device, a DNA diagnostic assay and a composition disclosed herein.

### DETAILED DESCRIPTION

Hidradenitis suppurativa (HS), also called acne inversa, is a chronic inflammatory skin disease. It affects apocrine gland-bearing skin, typically observed in the axillae (armpit), groin, and under the breasts. Its clinical presents as persistent or recurrent solid nodules and abscesses. Abscesses may present with purulent discharge, and after healing may produce scarring. Hidradenitis suppurativa has a significant psychological impact. Many patients with HS suffer from anxiety, depression, and have a negative body image. To date there are no approved medical treatments for HS.

As used herein, the terms “prevent” or “prevention” and other derivatives of the words, when used in reference to hidradenitis suppurativa, i.e., HS, refer to a reduced likelihood of HS in an individual receiving a given treatment relative to that of a similar individual at risk for HS but not receiving that treatment. As such, the terms “prevent” and “prevention” encompass a treatment that results in a lesser degree of HS than would be otherwise expected for a given individual. Efficacy for prevention of HS can be established through controlled studies, e.g., in which a subject is administered a treatment (e.g., an oral treatment) and another subject is administered a placebo. Under these circumstances, if the subject treated with the oral treatment displays less nodules and abscesses relative to the subject receiving the placebo, e.g., at least 5% less, at least 10% less, at least 15% less, at least 20% less, at least 25% less, at least 30% less, at least 35% less, at least 40% less, at least 45% less, at least 50% less or beyond, the treatment is effective for the prevention of HS.

As used herein, the terms “treat,” “treatment,” or “treating” refer to therapeutic treatments, wherein the object is to reverse, alleviate, ameliorate, inhibit, slow down or stop the progression or severity of a disease or condition, e.g., hidradenitis suppurativa or HS. The term “treating” includes reducing or alleviating at least one adverse effect or symptom of a disease or condition, e.g., HS. Treatment is generally “effective” if one or more symptoms are reduced. Alternatively, treatment is “effective” if the progression of a disease is reduced or halted. That is, “treatment” includes not just the improvement of symptoms, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality. For example, treatment is considered effective if the extent or amount of boil-like nodules, abscesses, purulent discharge, sinuses, or scarring is reduced, or the progression of boil-like nodules, abscesses, purulent discharge, sinuses, or scarring is slowed or halted. The term “treatment” of a disease also includes providing relief from the symptoms or side-effects of the disease (including palliative treatment). Treatment can involve administering a therapeutically effective amount of any one or combination of the compositions or anti-androgens disclosed herein. A “therapeutically effective amount” is an amount sufficient to provide an observable therapeutic benefit compared to HS left untreated in a subject or patient.

As used herein the term “comprising” or “comprises” is used in reference to compositions, methods, etc. refers to component(s) or method steps that are present in the method or composition, yet allows for the composition, method, etc. to also include unspecified elements.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Applicants disclose herein systems and methods for treating or preventing hidradenitis suppurativa (HS) in a subject in need thereof. The method includes the use of anti-androgens such as androgen receptor antagonists, androgen synthesis inhibitors, agents that counter the effect of androgens such as sex hormone-binding globulin (SHBG) stimulators, antigonadotropins, mineralocorticoids and glucocorticoids (anticorticotropins) suppressing androgen production in the adrenal gland, insulin sensitizing medications such metformin and vaccines and immunogens against androstenedione that reduce levels of testosterone and estrogen, some examples include ovandrotone albumin and androstenedione albumin, in a composition to treat or prevent hidradenitis suppurativa (HS). The anti-androgens may be present in amounts ranging from 0.1% to 100% of the total weight of the composition. These compositions can be administered topically, nasally, orally, or by injection. Compositions applied to a subject would alter androgen receptor function and subsequently treat or prevent hidradenitis suppurativa (HS). Other embodiments are described below.

In certain embodiments, the method for treating or preventing HS in a subject in need thereof comprises administering between 0.5 mgs to 800 mgs of an anti-androgen composition disclosed herein.

The methods disclosed herein may comprise a step of determining if a subject is at risk of developing HS by examining the occurrence of genetic variations within the subject. In certain embodiments, an occurrence of a genetic variation in the androgen receptor (AR) gene of the subject can be used to predict the likelihood if that subject will suffer from HS. In other embodiments, an occurrence of a genetic variation in a subject's androgen response elements of other genes under regulatory control of the androgen receptor can be used to predict the likelihood if that subject will suffer from HS.

The methods disclosed herein may comprise a step of determining if a subject is sensitive to anti-androgen treatment by examining the occurrence of genetic variations within the subject. In certain embodiments, a genetic variation in the androgen receptor (AR) gene of the subject can be used to predict the likelihood that an anti-androgen composition will treat or prevent hidradenitis suppurativa (HS). In other embodiments, an occurrence of a genetic variation in a subject's androgen response elements of other genes under regulatory control of the androgen receptor can be used to predict the likelihood that an anti-androgen composition will treat or prevent hidradenitis HS.

Many genetic variations in the androgen receptor (AR) gene can be used to predict the likelihood that a drug will treat or prevent HS but may include, the number CAG repeats in the first exon of AR gene. In certain embodiments of the present invention, a cut off value for the number of CAG repeats in the first exon of AR gene can be used to define a person with androgen sensitivity. In one embodiment the cut-off value for the number of CAG repeats the first exon of AR gene is 24.

In certain embodiments of the present invention a kit containing a DNA sample collection tool is envisioned. The genetic sample can be obtained by buccal swab, saliva, blood, or tissue samples. The genetic sample can also be obtained from a plucked hair sample. For example, a kit is disclosed for androgen sensitivity; the kit is an in-vitro diagnostic medical device intended to identify polymorphisms in the androgen receptor gene. The kit consists of a DNA collection device (buccal swab) and a DNA diagnostic assay (laboratory based). DNA samples are collected from a patient and mailed to a laboratory for processing.

In certain embodiments, the number of CAG repeats in the first exon of the AR gene is used as a genetic variant. In other embodiments, the variants in the promoter region of the AR are used as a genetic variant. In another embodiment, a shorter CAG repeat (compared to normal) may predict the likelihood that a drug will treat or prevent HS. In another embodiment, the length of the CAG repeat determines anti-androgen dosing for the treatment to increase the likelihood that a drug will treat or prevent HS.

Various aspects of the technology describe detecting androgen sensitivity in a subject by measuring polymorphisms in the androgen receptor gene. Androgen Receptor (AR) genetic variation refers to DNA genetic variations, expression of AR in specific tissue (RNA), including methylation analysis of AR (i.e., in the case of X-chromosome inactivation). Androgen sensitivity may also include DNA genetic variations in androgen response elements (ARE) of genes under the regulatory control of the AR. Examples of genes containing AREs include, but are not limited to, TMPRSS2 and ACE2. Polymorphisms in the androgen response elements can also be used to infer a subject has androgen sensitivity.

In certain embodiments of the present invention genetic variations in the androgen receptor (AR) gene or the promoter region of the AR can be used to predict a treatment response for HS. In another embodiment of the present invention genetic variations in the androgen receptor (AR) gene or the promoter region of the AR can be used to select a dosage of a treatment drug for HS. In certain embodiments genetic variations in the androgen receptor (AR) gene or the promoter region of the AR are single nucleotide polymorphisms (SNPs). In other embodiments SNPs that are associated with AR expression or function are used.

Examples of genetic variations in the androgen receptor (AR) gene or the promoter region of the AR, or are associated with AR expression or function include but are not limited to rs137852591, rs104894742, rs1057518177, rs1057521121, rs1057521122, rs1057523747, rs1064793480, rs1064793645, rs1064794065, rs1064794069, rs1064795250, rs1085307685, rs1085307962, rs12014709, rs1204038, rs1337080, rs137852562, rs137852563, rs137852564, rs137852565, rs137852566, rs137852567, rs137852568, rs137852569, rs137852570, rs137852571, rs137852572, rs137852573, rs137852574, rs137852575, rs137852576, rs137852577, rs137852578, rs137852579, rs137852580, rs137852581, rs137852582, rs137852583, rs137852584, rs137852585, rs137852586, rs137852587, rs137852588, rs137852589, rs137852590, rs137852592, rs137852593, rs137852594, rs137852595, rs137852596, rs137852597, rs137852598, rs137852599, rs137852600, rs137852601, rs1800053, rs201934623, rs2361634, rs5031002, rs5918757, rs6152, rs6624304, rs750324117, rs754201976, rs755226547, rs759327087, rs864622007, rs869320731, rs869320732, rs878853033, rs886039558, rs886041050, rs886041128, rs886041129, rs886041130, rs886041131, rs886041132, rs886041133, rs886041352, rs9332969, rs9332971 or any combination thereof.

Androgen sensitivity may also include DNA genetic variations in androgen response elements (ARE) of genes under the regulatory control of the AR. Examples of genes containing AREs include, but are not limited to, TMPRSS2, furin and ACE2.

Many genetic variations in the androgen receptor (AR) gene can be used to predict the likelihood that a drug will treat or prevent HS and may include the number of CAG repeats in the first exon of AR gene. In another embodiment, the number GGN (polyglycine) repeats in the first exon of AR gene can be used to predict the likelihood that a drug will treat or prevent HS. In certain embodiments of present invention the number of GGN repeats is between 10 and 30. In another embodiment, the number of GGN repeats can be used to define a person with androgen sensitivity. In one embodiment the ratio of CAG to GGN repeats in the first exon of AR gene is used to define a person with androgen sensitivity. In some embodiments the number of CAG, GGN, or the ration of CAG/GGN is used to predict a treatment response or choose a dosage of a drug or treatment regimen.

In certain embodiments, a method of treating or preventing HS consists of administering an anti-androgen to a patient where there anti-androgen is selected among: cyproterone acetate, megestrol acetate, chlormadinone acetate, spironolactone, medrogestone, oxendolone, osaterone, bifluranol acetate, finasteride, dutastride, flutamide, bicalutamide, nilutamide, topilutamide, enzalutamide, apalutamide, dienogest, drospirenone, medrogestone, nomegestrol acetate, promegestone, trimegestone, ketoconazole, abiraterone acetate, seviteronel, aminoglutethimide, epristeride, alfaestradiol, isotretinoin, saw palmetto, marijuana, cannabinoids, darolutamide, EZN-4176, AZD-3514, and AZD-5312, apatorsen, galeterone, ODM-2014, TRC-253, BMS-641988, proxalutamide (GT0918, CAS #: 1398046-21-3), luteinizing hormone-releasing hormone (LH-RH), follicle-stimulating hormone (FSH), triptorelin pamoate, docetaxel, diethylstilbestrol, tadalafil, silodosin, tamsulosin hydrochloride, naftopidil, solifenacin succinate, tamsulosin, tamsulosin hydrochloride, alfuzosin hydrochloride, prazosin hydrochloride, doxazosin, doxazosin mesylate, solifenacin succinate, allylestrenol, benzydamine hydrochloride, cefatrizine, chlormadinone acetate, flavoxate hydrochloride, gestonorone caproate, indoramin hydrochloride, mepartricin, oxybutynin chloride, phenoxybenzamine hydrochloride, terazosin, terazosin hydrochloride, or degarelix. As explained herein, the method of treatment can involve administration of a composition that includes an anti-androgen as an ingredient of the composition. The anti-androgen can be present in percent amounts ranging from 0% to 100%.

In certain embodiments, a method of treating or preventing HS consists of administering an anti-androgen to a patient where the anti-androgen is proxalutamide. Proxalutamide can be present in percent amounts ranging from 0.1% to 10%. Proxalutamide can be present in percent amounts ranging from 0.1% to 1%. Proxalutamide can be present in percent amounts ranging from 1% to 10%. Proxalutamide can be present in percent amounts ranging from 0.1% to 50%. Proxalutamide can be present in percent amounts ranging from 10% to 50%. In some embodiments, the composition consists essentially of proxalutamide. In embodiments wherein the composition comprises proxalutamide, other anti-androgens may be present in amounts ranging from 0.1% to 99.9%, 0.1% to 50%, 10% to 50%, 20% to 40%, 30% to 50%, 1% to 10%, or 0.1% to 5%. In another embodiment proxalutamide is delivered orally at a dosage of 50-400 mg once daily. In another embodiment proxalutamide is delivered orally at a dosage of 200 mg once daily.

In certain embodiments, a method of treating or preventing HS consists of administering an anti-androgen to a patient where the anti-androgen is GT0918. GT0918 can be present in percent amounts ranging from 0.1% to 10%. GT0918 can be present in percent amounts ranging from 0.1% to 1%. GT0918 can be present in percent amounts ranging from 1% to 10%. GT0918 can be present in percent amounts ranging from 0.1% to 50%. GT0918 can be present in percent amounts ranging from 10% to 50%. In some embodiments, the composition consists essentially of GT0918. In embodiments wherein the composition comprises GT0918, other anti-androgens may be present in amounts ranging from 0.1% to 99.9%, 0.1% to 50%, 10% to 50%, 20% to 40%, 30% to 50%, 1% to 10%, or 0.1% to 5%. In another embodiment GT0918 is delivered orally at a dosage of 50-400 mg once daily. In another embodiment GT0918 is delivered orally at a dosage of 200 mg once daily.

In certain embodiments, a method of treating or preventing HS involves administering an agent that counters the effect of androgens such as sex hormone-binding globulin (SHBG) stimulators. As explained herein, the method of treatment can involve administration of a composition that includes said agent as an ingredient of the composition. The agent can be present in percent amounts ranging from 0% to 100%.

In certain embodiments, a method of treating or preventing HS involves administering an antigonadotropin. As explained herein, the method of treatment can involve administration of a composition that includes an antigonadotropin as an ingredient of the composition. The antigonadotropin can be present in percent amounts ranging from 0% to 100%.

In certain embodiments, a method of treating or preventing HS involves administering a mineralocorticoid and/or a glucocorticoid (e.g., anticorticotropin) suppressing androgen production in the adrenal gland. As explained herein, the method of treatment can involve administration of a composition that includes a mineralocorticoid and/or a glucocorticoid as an ingredient of the composition. The mineralocorticoid and/or a glucocorticoid can be present in percent amounts ranging from 0% to 100%.

In certain embodiments, a method of treating or preventing HS involves administering an insulin sensitizing medication (e.g., metformin). As explained herein, the method of treatment can involve administration of a composition that includes an insulin sensitizing medication as an ingredient of the composition. The insulin sensitizing medication can be present in percent amounts ranging from 0% to 100%.

In certain embodiments, a method of treating or preventing HS involves administering a vaccine or immunogen against androstenedione that reduces the level of testosterone or increases estrogen. Examples of such vaccines and immunogens can be ovandrotone albumin and androstenedione albumin. As explained herein, the method of treatment can involve administration of a composition that includes said vaccine or immunogen as an ingredient of the composition. The vaccine or immunogen can be present in percent amounts ranging from 0% to 100%.

In certain embodiments, a genetic variation in the AR gene is used as a predictor of anti-androgen treatment response. In certain embodiments, a genetic variation in the AR gene is used to guide selection of the appropriate anti-androgen treatment. In certain embodiments, a genetic variation in the AR gene is used as a predictor of anti-androgen treatment response for HS. In certain embodiments, a genetic variation in the AR gene is used to guide selection of the appropriate anti-androgen treatment for HS.

In certain embodiments, an anti-androgen can mean a treatment or composition that promotes the production of estrogen. In another embodiment estrogen is the treatment. In another embodiment, the composition further comprises estrogen or agents that promote the production of estrogen. Examples of agents that promote the production of estrogen include, but are not limited to, estrogen, estradiol, Premarin (conjugated estrogens), medroxyprogesterone acetate, Provera, medroxyprogesterone, Vivelle-Dot (estradiol patch).

Any of the compositions disclosed herein can include other ingredients (e.g., carrier agents) to facilitate use or administration of the composition via injection, oral administration, nasal administration, topical administration, etc. For instance, a composition can include a carrier or delivery vehicle optimized for delivery of the composition to the skin. As another example, a composition can be formulated to be released using several different formulations or release methods including time release, creams, ointments, sprays, capsules, antiperspirants, or other release methods. Capsules or vehicles that encapsulate the composition can include, but are not limited to, liposomes, non-ionic liposomes, niosomes, novasome I, erythromycin-Zn complex, microspheres, nanoparticles, solid lipid nanoparticles, and nanoemulsions. In some embodiments, this can include a gel or foam.

In certain embodiments, the treatments or compositions can be administered by inhalation, oral, nasal, or injection. In certain embodiments, the treatments or compositions can be administered by nebulization or vaping. In certain embodiments, the treatments or compositions can be administered systemically in oral, intravenous injection, subcutaneous injection. In certain embodiments, the treatments or compositions can be administered topically to a subject. Topical administration may include applying an anti-androgen composition into a HS lesion on the subject.

It should be noted that any of the ingredients disclosed herein can be used in combination with any one or combination of other ingredients (e.g., an anti-androgen can be used with an agent that counters the effect of androgens, an antigonadotropin can be used with a mineralocorticoid, an insulin sensitizing medication can be used with an anti-androgen and a mineralocorticoid, etc.).

In some embodiments, provided herein is an anti-androgen formulated with a carrier or delivery vehicle optimized for delivery of the anti-androgen treatment to the skin. An anti-androgen can be released using several different formulations or release methods including time release, creams, ointments, sprays, capsules, or other release methods. Capsules or vehicles that encapsulate the anti-androgen can include, but are not limited to, liposomes, non-ionic liposomes, niosomes, novasome I, erythromycin-Zn complex, microspheres, nanoparticles, solid lipid nanoparticles, and nanoemulsions. In some embodiments, this can include a gel or foam.

In some embodiments, the anti-androgen is combined with an anti-inflammatory agent, such as an NSAID.

In some embodiments, the anti-androgen is combined with an anti-bacterial agent, such as an azithromycin.

In some embodiments, the anti-androgen is combined with aspartame.

In some embodiments, the anti-androgen treatment is administered orally.

In other embodiments, the anti-androgen treatment is administered nasally.

In other embodiments, the anti-androgen treatment is administered by inhalation.

In other embodiments, the anti-androgen treatment is administered topically via the skin.

In other embodiments, the anti-androgen treatment is administered intramuscularly or intravenously.

Any of the aforementioned anti-androgens can be used routinely, e.g., once daily, twice daily, every other day, once a week. Additionally, it is envisioned that an anti-androgen can be used before, during (short period) or after an HS episode, e.g., an anti-androgen treatment might be used for 1, 2, 3, 4, and more months after an HS episode.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

The recitation of ranges of values herein is merely intended to serve as a shorthand method of referring individually to each separate value falling within the range (the range including the end points of the range). Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that can have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

### EXAMPLES

### Example 1: Study of Proxalutamide for the Treatment of HS

Overview: Four subjects, 2 males and 2 females, were recruited to the Proxalutamide HS study. At baseline (day 0) all subjects were diagnosed by a dermatologist with HS Hurley Stage 3. All subjects presented HS in the axilla region. Two subjects (1 male and 1 female) were treated for 7 days with Proxalutamide monotherapy 200 mg daily and two subjects were treated for 7 days with Proxalutamide placebo monotherapy 200 mg daily

Efficacy Endpoint: Hurley Stage following 7 days of treatment.

Formula: Proxalutamie 200 mg q.d.

Results: Following 7 days of treatment with Proxalutamide 200 mg q.d., all four subjects returned to the clinic to be assessed by a dermatologist. On day 7 both subjects treated with Proxalutamie were diagnosed by a dermatologist with HS Hurley Stage 1. No adverse events were reported by the subjects. On day 7 both subjects treated with Proxalutamie placebo were diagnosed by a dermatologist with HS Hurley Stage 3. No adverse events were reported by the subjects.

Conclusion: In our study, Proxalutamide reduced the severity of HS within 7 days without any adverse events when compared to the placebo

1. A method for treating or preventing hidradenitis suppurativa in a subject in need thereof, wherein the method comprises administering a therapeutically effective amount of an anti-androgen composition to the subject, wherein the composition comprises proxalutamide.

2. The method according to claim 1, wherein the composition further comprises other anti-androgens selected from the group consisting of androgen receptor antagonists, androgen synthesis inhibitors, sex hormone-binding globulin (SHBG) stimulators, antigonadotropins, mineralocorticoids, glucocorticoids, insulin sensitizing medications, and vaccines or immunogens against androstenedione.

3. The method according to claim 2, wherein the glucocorticoids and mineralocorticoids include anticorticotropin.

4. The method according to claim 2, wherein the insulin sensitizing medication include metformin.

5. The method according to claim 2, wherein the vaccines or immunogens against androstenedione include ovandrotone albumin and androstenedione albumin.

6. The method according to claim 2, wherein the other anti-androgens are present in an amount ranging from 0.1% to 99.9% of the total weight of the composition.

7. The method according to claim 1, wherein proxalutamide is present in an amount ranging from 0.1% to 50% of the total weight of the composition.

8. The method according to claim 1, wherein the composition consists essentially of proxalutamide.

9. The method according to claim 1, wherein the method further comprises determining if the subject is sensitive to anti-androgen treatment by examining an occurrence of genetic variations in the subject's androgen receptor gene or in the subject's androgen response elements of other genes under regulatory control of the androgen receptor.

10. The method of claim 9, wherein the genetic variations occur in the number CAG repeats in the first exon of the androgen receptor gene, the number GGN repeats in the first exon of the androgen receptor gene or in the promoter region of the androgen receptor gene.

11. The method of claim 9, wherein the genetic variation occurs in the subject's androgen response elements for TMPRSS2, furin and ACE2.

12. The method of claim 9, wherein the occurrence of genetic variations are in the androgen receptor gene or promoter region of the androgen receptor gene, and wherein the genetic variations are rs137852591, rs104894742, rs1057518177, rs1057521121, rs1057521122, rs1057523747, rs1064793480, rs1064793645, rs1064794065, rs1064794069, rs1064795250, rs1085307685, rs1085307962, rs12014709, rs1204038, rs1337080, rs137852562, rs137852563, rs137852564, rs137852565, rs137852566, rs137852567, rs137852568, rs137852569, rs137852570, rs137852571, rs137852572, rs137852573, rs137852574, rs137852575, rs137852576, rs137852577, rs137852578, rs137852579, rs137852580, rs137852581, rs137852582, rs137852583, rs137852584, rs137852585, rs137852586, rs137852587, rs137852588, rs137852589, rs137852590, rs137852592, rs137852593, rs137852594, rs137852595, rs137852596, rs137852597, rs137852598, rs137852599, rs137852600, rs137852601, rs1800053, rs201934623, rs2361634, rs5031002, rs5918757, rs6152, rs6624304, rs750324117, rs754201976, rs755226547, rs759327087, rs864622007, rs869320731, rs869320732, rs878853033, rs886039558, rs886041050, rs886041128, rs886041129, rs886041130, rs886041131, rs886041132, rs886041133, rs886041352, rs9332969, rs9332971 or any combination thereof.

13. The method of claim 1, wherein the amount of proxalutamide in the composition ranges from 50-400 mgs.

14. The method of claim 1, wherein the composition is administered topically as a gel, foam or lotion into a hidradenitis suppurativa lesion on the subject, or
the composition is administered orally to the subject, or
the composition is administered in an amount ranging from 0.5 mg to 800 mg to the subject.

15. (canceled)

16. (canceled)

17. An anti-androgen composition for treating or preventing hidradenitis suppurativa in a subject in need thereof, wherein the composition comprises a therapeutically effective amount proxalutamide.

18. The composition according to claim 17, wherein the composition further comprises an anti-inflammatory agent or an anti-bacterial agent, or
the composition further comprises other anti-androgens selected from the group consisting of androgen receptor antagonists, androgen synthesis inhibitors, sex hormone-binding globulin (SHBG) stimulators, antigonadotropins, mineralocorticoids, glucocorticoids, insulin sensitizing medications, and vaccines or immunogens against androstenedione.

19. (canceled)

20. The composition according to claim 17, wherein the composition further comprises aspartame.

21. The composition according to claim 17, wherein the composition further comprises carriers or delivery vehicles selected from capsules liposomes, non-ionic liposomes, niosomes, novasome I, erythromycin-Zn complex, microspheres, nanoparticles, solid lipid nanoparticles and nanoemulsions.

22. The composition of claim 17, wherein the composition further comprises agents that promote the production of estrogen, wherein the agents are selected from estrogen, estradiol, conjugated estrogens, medroxyprogesterone acetate, Provera and medroxyprogesterone.

23. (canceled)

24. (canceled)
